(19) United States
(12) Patent Application Publication
(10) Pub. No.: US 2004/0166149 A1
(43) Pub. Date: Aug. 26, 2004

(54) Optimizing giant unilamellar vesicle (GUV) growth in a physiological buffer and artificial cell based biosensor

(76) Inventor: Juyang Huang, Lubbock, TX (US)

Correspondence Address: JONES, TULLAR & COOPER, P.C., P.O. BOX 2266 EADS STATION, ARLINGTON, VA, 22202

(21) Appl. No.: 10/682,775

(22) Filed: Oct. 10, 2003

Related U.S. Application Data
(60) Provisional application No. 60/417,140, filed on Oct. 10, 2002.

(51) Int. Cl. A61K 9/127

(52) U.S. Cl. 424/450

(57) ABSTRACT

The yield of giant unilamellar vesicles (GUVS) is greatly increased by influencing the characteristics of the lipid film. Lipid films are formed in containers made from hydrophobic material that have a low affinity for hydrated lipid films, preferably TEFLON test tubes. Additionally, the lipid film is dried rapidly using a vortex-drying process. The GUVs that are formed may be incorporated into an artificial cell based biosensor. The biosensor includes an addressable array containing a plurality of host cavities for holding the GUVs, preferably functionalized GUVs.

### CROSS-REFERENCE TO RELATED APPLICATION

This application claims the benefit, under 35 U.S.C. 119(e), of U.S. Provisional Application No. 60/417,140 filed Oct. 10, 2002, the contents of which are incorporated herein by reference.

### BACKGROUND OF INVENTION

1. Field of Invention

The present invention relates generally to a method for increasing the yield of giant unilamellar vesicles, GUVs. The GUVs are preferably disposed within an addressable array to fabricate artificial cell based biosensors.

2. Description of Related Art

Cell plasma membranes provide the native environment for transmembrane proteins, ion channels, and receptors to carry out their biological function. In recent years, biosensor systems based on planar supported lipid bilayers have been actively researched. However, because the lipid bilayers are only about 1-2 nm from the solid substrate surfaces, a common problem is that integral membrane proteins are not laterally mobile in these systems. Because many membrane proteins and ion channels protrude from the membrane surface more than 2 nm, the geometry is not favorable for them to function normally.

Synthetic liposomes, particularly giant unilamellar vesicles, GUVs, have the same lipid bilayer structure as cell plasma membranes, and they can be prepared to match the size and composition of real cell membranes. GUVs have a diameter of 10 to 100 μm and are good models of cell membranes. GUVs can be used to study membrane domains and phase separation in multi-component liposomes by optical microscopy. By selectively incorporating receptors, ion channels and antibodies on GUVs, one can construct artificial cells, such as functionalized GUVs, which closely mimic real cell membranes. These artificial cells, equipped with proper fluorescent probes and electrodes, can be used to detect the presence of ligands, antigens, and nonspecific ion channel blockers, such as toxic agents, ligand inhibitors, and other molecules.

Prior methods of making GUVs in a physiological buffer consist of five primary steps which allow for a smooth lipid film to be gently dried onto glass test tubes and then slowly hydrated. The five steps are generally as follows:

Step 1: Glass tubes are selected and cleaned in order to prepare for lipid deposition.

Step 2: 50 uL of 10 mg/mL lipid in 2:1 CHCl₃/MeOH solution is added to the tube.

Step 3: The solution is slowly dried onto the glass in a rotary evaporator and then dried for 6 hours under a strong vacuum.

Step 4: The dry film is slowly hydrated by a stream of water saturated N₂ at 45° C.

Step 5: A buffer solution is gently added to the hydrated film and allowed to incubate overnight.

However, the majority of lipids formed in this process are multi-lamellar vesicles and a very small number of GUVs are produced in this method. Therefore, a method which increases the yield rate of GUVs would be advantageous.

### BRIEF SUMMARY OF THE INVENTION

The present invention is directed to a method for increasing the yield rate of giant unilamellar vesicles, GUVs. First, a container made from a hydrophobic material is selected and cleaned. The hydrophobic material promotes spreadability of a lipid solution, in an organic solvent, on its surface for the formation of a continuous lipid film. The hydrophobic material also has a low affinity for hydrated lipids so that a hydrated lipid film is easily detached from the container surface, thus increasing the hydrated lipid film surface area exposed to the buffer solution. Finally, the hydrophobic material can have a rough surface upon which the lipid films are formed. The preferred container made from the hydrophobic material is a TEFLON test tube, which is preferred over glass test tubes due to their surface characteristics. Second, a lipid solution is added to the container. Third, a lipid film is produced by vortex-drying the lipid solution while under vacuum to produce a dried lipid film. A vortex-drying method produces more GUVs than traditionally produced with a rotary-evaporator. Additionally, the vortex-drying method is much faster than the rotary-evaporator method. Further drying may be needed to remove residue solvent. Fourth, the dried lipid film is slowly hydrated, preferably by a stream of water saturated nitrogen, resulting in a hydrated lipid film. Finally, a buffer solution is gently added to the hydrated lipid film and allowed to incubate.

The present method is an improvement over prior methods for making GUVs. This method increases the surface area of the lipid film and modifies the characteristics of the lipid film. First, after adding the buffer solution, the hydrated lipid films quickly detach from the surface of the container due to the hydrophobic nature and become suspended in the buffer solution. Thus, GUVs can grow from both sides of the lipid film. In previous methods using glass tubes, the lipid films stick to the glass and GUVs can only grow from the side facing the buffer solution. Second, the surface roughness of the lipid film helps GUV formation. The surface of the lipid film facing the preferred TEFLON test tube is molded into the surface of the test tube, which is typically rough. On the other hand, the lipid film surface facing the buffer solution is also rough due to vortexing. In contract with the prior methods, the surface facing the buffer solution is quite smooth. Third, the internal structure of the lipid film made by the present method may be less compact than that made by the prior method, which also helps GUV formation.

The present method can produce a large quantity of GUVs, which is particularly suitable for the production of artificial cell based biosensors. This type of biosensor has the potential to be used for high throughput detection of chemical and biological agents, environmental monitoring, clinic diagnosis, and ion channel drug and ligand inhibitor screening. By combining nanofabrication, liposome technology and molecular biology a versatile artificial cell based biosensor is fabricated. The preferred biosensor includes GUVs that are functionalized and disposed on an addressable array containing host cavities. In a preferred embodiment, the biorecognition surfaces are on the top of the GUVs, free from the substrate surfaces. Since the geometry of the GUVs is very similar to real cell surfaces, an ideal environment is provided for membrane receptors and ion channels. Furthermore, host cavities formed in the biosensor isolate each detection GUV from another, which can greatly reduce the cross-site contamination. Thus, a single biosensor has the potential to accommodate hundreds or even thousands of GUV detection cells.

### BRIEF DESCRIPTION OF THE DRAWING(S)

The features and advantages of the present invention will become apparent from the following detailed description of a preferred embodiment thereof, taken in conjunction with the accompanying drawings, in which:

FIG. 1 is a diagram showing the preferred device used to accomplish the vortex-drying;

FIG. 2 is a picture of a differential interference contract (DIC) image of a sample of GUVs prepared by the present method; and

FIG. 3 shows the GUVs in the host cavities of the biosensor.

### DETAILED DESCRIPTION OF THE INVENTION

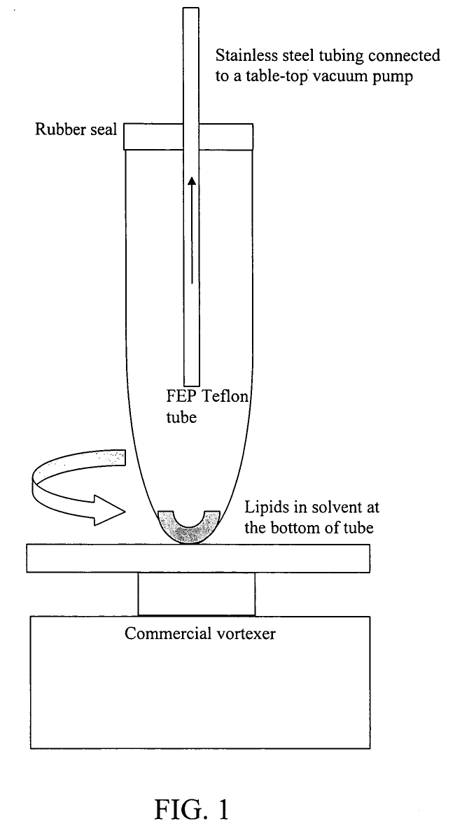

The yield rate of giant unilamellar vesicles, GUVs, is increased by more than 10 times by forming the GUVs according to the following process. First, a container made from a hydrophobic material is selected and cleaned. The hydrophobic material promotes spreadability of a lipid solution on its surface for the formation of a continuous lipid film. The hydrophobic material also has a low affinity for lipids so that a hydrated lipid film is easily detached from the container surface. Finally, some hydrophobic material has a rough surface upon which the lipid films are formed. The preferred hydrophobic material is a fluorocarbon polymer, such as polytetrafluoroethylene or TEFLON. The preferred container may be any shape or size and may include test tubes, chambers, bottles or cells. TEFLON test tubes are preferred over glass test tubes due to their surface characteristics. Second, a lipid solution is added to the container. A wide variety of lipids may be used, including, but not limited to, phosphotidylcholines (PCs), phosphotidylethanolamines (PEs), cholesterol, anionic lipids, and glycolipids. Preferred lipids include 1-palmitoyl-2-oleyol-3-phosphatidylcholine (POPC) and 1-palmitoyl-2-oleyol-3-phosphatidylglycerol (POPG). The lipid solution may contain a variety of solvents. In a preferred lipid solution, an organic solvent such as chloroform/methanol solvent is used. Third, a lipid film is produced by vortex-drying the lipid solution while under vacuum to produce a dried lipid film. FIG. 1 shows a diagram of a preferred device used for the vortex-drying step of the present method. The preferred FEP TEFLON test tube, which is covered with a rubber seal, is connected to a vacuum pump through a stainless steel tubing. The lipid solution collects at the bottom of the test tube. In order to form the dried lipid film, the test tube is subjected to vortexing using any commercial vortexer. Further drying may be needed to remove residue solvent. Fourth, the dried lipid film is slowly hydrated, preferably by a stream of water saturated nitrogen, resulting in a hydrated lipid film. Finally, a buffer solution is gently added to the hydrated lipid film and allowed to incubate. A preferred buffer solution includes pH buffers, such as a pH buffer of 1,4-piperazine diethane sulfonic acid, 1.5 sodium salt (PIPES), salt, such as KCl, and EDTA. The buffers solution is preferably warm, at approximately 35° C. to avoid cold shock to the lipid film.

The GUVs that are produced may be further used to fabricate artificial cell based biosensors by disposing the GUVs in an addressable array containing a plurality of host cavities. By combining micro-fabrication, liposome technology and molecular biology a versatile artificial cell based biosensor is fabricated. The biosensor is formed from any suitable material, preferably a glass substrate. The biosensor may include a non-binding surface coating, preferably a fluorocarbon polymer, such as TEFLON. Host cavities are formed in the substrate using known etching techniques. In a preferred embodiment, the host cavities are coated with a lipid bilayer, preferably using small unilamellar vesicles, SUVs, which fuse to the substrate surface. This lipid bilayer greatly reduces the undesirable surface effects of the substrate on the GUVs. The GUVs are loaded into the host cavities by any suitable technique, including, but not limited to, optical trapping, micro-pipetting, and microfluidic delivery. The GUVs are held in the host cavities by their geometric shape. The preferred biosensor includes GUVs that are functionalized and disposed on an addressable array. The GUVs may be functionalized with bio-recognition molecules including, but not limited to, receptors, ion channels, antibodies, and synthetic bio-recognition molecules. In a preferred embodiment, the bio-recognition surfaces are on the top of the GUVs, free from the substrate surfaces.

Example Procedure for Growing GUVs using Vortex Drying Method with Teflon Tubes:

In a preferred method for making GUVs, a lipid film is formed from 50 micro-liters of lipid solution including 9:1 POPC/POPG (1-palmitoyl-2-oleyol-3-phosphatidylcholine/1-palmitoyl-2-oleyol-3-phosphatidylglycerol), 10 mg/mL in 2:1 Chloroform/Methanol with 1% Rhodamine-PE fluorescence label in a TEFLON FEP centrifuge tube (50 mL size, Nalgene Oak Ridge). POPG was added due to its negative charge to enhance the unilamellarity by creating an added repulsion between the lipid films as they peel away from each other. To enhance lipid coverage on the test tube, approximately 300 micro-liters of 2:1 Chloroform/ Methanol solution was added.

A lipid film was created on the bottom of the TEFLON FEP tube by attaching a table-top mechanical vacuum pump hose to the test tube and drying the tube with vacuum while vortexing the sample. This usually takes less than a minute to produce a dried lipid film. The vortexing was achieved using a Fisher Vortex Genie 2 from Fisher Scientific at the highest setting.

Residue solvent was removed by connecting the sample in a desiccator to a strong vacuum until pressures is dropped below 20 mTorr. This takes about 3 to 6 hours, depending on the number of tubes drying on the system.

The dried lipid film was then hydrated in the tube with H₂O Saturated N₂ gas at 45° C. for 30 minutes. This step seems least crucial, but optimal a hydrated lipid film is produced anywhere between 20 and 40 minutes.

The tube was then gently filled with a warm buffer solution including 5 mM 1,4-piperazine diethane sulfonic acid, 1.5 sodium salt (PIPES), 100 mM KCl, and 1 mM EDTA at approximately 35° C. to avoid cold shock to the hydrated lipid film. The amount of warm buffer solution was such that it covered the lipid film deposition area. Finally, the lipid films were incubated overnight at 37° C.

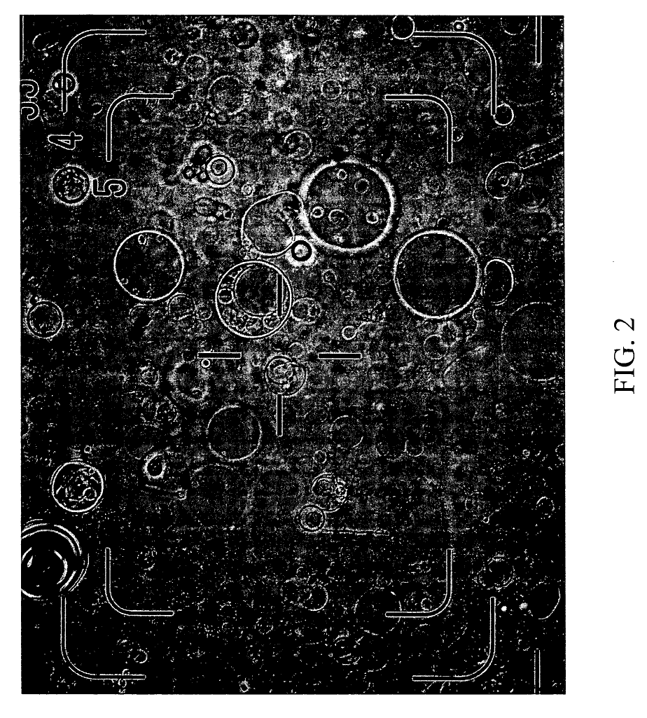

Lipid cluster formed in the buffer solution. Small amounts of the lipid clusters were micropipetted, 10-50 micro-liters, depending on the desired amount to put on a slide, and deposited on a viewing slide with parafilm spacers (Laboratory Parafilm from American National). GUVs can be viewed anywhere on the slide, but many are seen on the bottom of the sample. Optimally the sample is taken from just above the lipid cluster in the buffer solution, as the detachable vesicles are generally found floating around the lipid cluster. FIG. 2 shows a differential interference contrast (DIC) image of a sample containing GUVs prepared by this process. The length of each black bar at the center of FIG. 2 is 50 microns.

### Example Artificial Cell Based Biosensor

A biosensor containing an addressable array of small host cavities of 30-50 micro-meter size with proper surface coatings is made on a piece of a glass slide by known micro-fabrication techniques. The glass slide is preferably coated with a non-binding surface material, preferably polytetrafluorethylene or TEFLON, since lipids do not stick to this type of material.

Coating of a single lipid bilayer on the surface of host cavities

The slides are incubated in a suspension of small unilamellar vesicles (SUVs). SUVs are prepared by extruding multi-lamellar liposomes through tiny pores of a polycarbonate membrane. An example of the process is found in MacDonald, R. C. et al, 1991, “Small volume extrusion apparatus for preparation of large, unilamellar vesicles”, Biochem Biophys Acta 1061: 297-303. SUVs of diameter 50-100 nm have a strong tendency to fuse to the exposed glass surfaces and form a single bilayer. After incubation, excess SUVs are washed away with buffer.

Prepare GUVs in a physiological buffer

Multi-component GUVs in physiological buffers are prepared using the novel method presented herein. A preferred method of making the GUVs has been discussed in detail in the previous example.

Adding bio-regognition molecules to GUVs

GUVs are functionalized by adding receptors, antibodies, or ion channels to the GUVs. Bio-recognition molecules may be added using any known technique including, but not limited to, fusing GUVs with SUVs containing functional molecules.

Loading GUVs to host cavities

A variety of techniques may be used to load the GUVs in each host cavity including optical trapping, micro-pipetting, and microfluidic delivery. Since the host cavities form a regular array, this step is preferably automated. Note that cell surface and host cavity surface are separated by a lipid bilayer coating, which greatly reduces the undesirable surface effects of substrate on the cells. The cells are held in the cavity by its geometric shape.

Making multi-functional biosensors

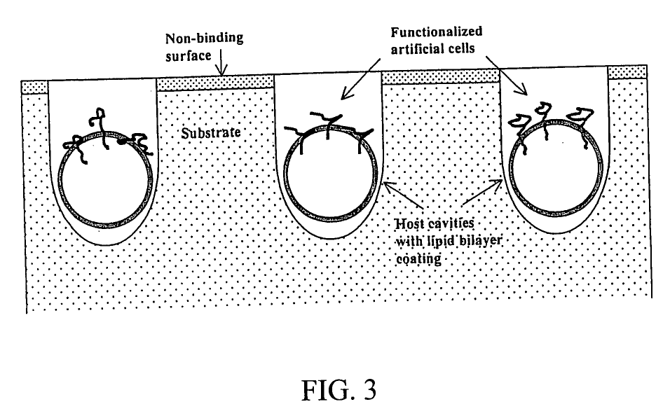

Many artificial cells with different functionalities are made on the same biosensor. The host cavities also serve to partition chambers to keep the functionalized agents inside each host cavity. FIG. 3 shows the preferred glass slide including host cavities with the functionalized GUVs.

Detection

A testing solution containing target molecules is introduced to the biosensor. The primary detection method is fluorescent microscopy. Fluorescent intensity, spectrum, anisotropy, and time-resolved fluorescent decay are recorded and analyzed. In the future, electrodes and other micro detection devices can be added.

Although the present invention has been disclosed in terms of a preferred embodiment, it will be understood that numerous additional modifications and variations could be made thereto without departing from the scope of the invention as defined by the following claims.

What is claimed is:

1. A method for making giant unilamellar vesicles comprising,
a) adding a lipid solution to a container made from a hydrophobic material.
b) vortex-drying said lipid solution to produce a dried lipid film;
c) hydrating the dried lipid film to produce a hydrated lipid film; and
d) adding a buffer solution to said hydrated lipid film.

2. The method of claim 1, whereby said container is selected from the group comprising a test tube, a chamber, a bottle and a cell.

3. The method of claim 1, whereby said material is a fluorocarbon polymer.

4. The method of claim 3, whereby said material is polytetrafluoroethylene.

5. The method of claim 1, whereby said lipid solution includes lipids selected from the group comprising phosphotidylcholines, phosphotidylethanolamines, cholesterol, anionic lipids, and glycolipids.

6. The method of claim 1, whereby said lipid solution includes a solvent selected from the group comprising chloroform and methanol.

7. The method of claim 1, whereby said step of hydrating includes subjecting said dried lipid film to a stream of water saturated with nitrogen.

8. The method of claim 1, whereby said buffer solution is selected from the group comprising pH buffers, salt, and EDTA.

9. The method of claim 1, further including incubating said hydrated lipid film in said buffer solution.

10. The method of claim 1, whereby said step of vortex-drying includes subjecting said lipid solution to a vacuum and vortexing.

11. An artificial cell based biosensor comprising,
a) an addressable array having a plurality of host cavities; and
b) a plurality of giant unilamellar vesicles disposed in said plurality of host cavities.

12. The artificial cell based biosensor of claim 11, whereby said addressable array further includes a non-binding surface coating.

13. The artificial cell based biosensor of claim 12, whereby said non-binding surface coating is polytetrafluoroethylene.

14. The artificial cell based biosensor of claim 11, whereby said plurality of host cavities are fabricated on a solid substrate.

15. The artificial cell based biosensor of claim 14, whereby said plurality of host cavities are coated with a lipid bilayer.

16. The artificial cell based biosensor of claim 15, whereby said lipid bilayer is formed by fusing small unilamellar vesicles to said solid substrate.

17. The artificial cell based biosensor of claim 11, whereby said plurality of giant unilamellar vesicles are functionalized.

18. The artificial cell based biosensor of claim 17, whereby said plurality of giant unilamellar vesicles are functionalized with bio-recognition molecules.

19. The artificial cell based biosensor of claim 18, whereby said bio-recognition molecules are selected from the group comprising receptors, ion channels, antibodies, and synthetic bio-recognition molecules.
